(19) United States
(12) Patent Application Publication
(10) Pub. No.: US 2009/0165806 A1
(43) Pub. Date: Jul. 2, 2009

(54) Straight jacket

(76) Inventor: Joel G. Tucker, Los Angeles, CA (US)

Correspondence Address: TODD S. PARKHURST;HUGHES SOCOL PIERS RESNICK & DYM LTD., THREE FIRST NATIONAL PLAZA, 70 WEST MADISON- SUITE 4000, CHICAGO, IL, 60602, US

(21) Appl. No.: 12/006,373

(22) Filed: Jan. 2, 2008

(51) Int. Cl. A61F 5/37

(52) U.S. Cl. 128/875

(57) ABSTRACT

A straitjacket is disclosed, which is adapted to fit wearers of more than one clothing size. The straitjacket has a jacket torso body comprising at least two panels, each panel terminating at a lower edge located adjacent to but below the wearer's underarms. Sleeves extend from the torso body to distal ends, and each sleeve has a length greater than the length of the wearer's arms. The distal end of each sleeve includes a fastening device to permit the sleeve to be fastened to the opposite sleeve and to a belt by suitable fasteners. An annular jacket collar is located above the jacket torso body, and a strap extends downwardly from the collar and under the wearer's crotch. A belt strap is adapted to be secured about the wearer's mid rib cage and is further adapted to be attached to the chest strap at a posterior position; and, if desired, at an anterior position.

### BACKGROUND OF THE INVENTION

Straitjackets are garments intended to restrain a potentially violent patient from use of his or her arms, so as to prevent injury to himself or herself or to others. Traditional straitjackets have torso bodies which extend down to, or finish, at or near the hip level. Sleeves attached to the torso body each have lengths which extend beyond the wearer's fingertips. Straps or other fasteners are attached to the distal ends of each sleeve. Once the patient's arms are inside the sleeves and the back is fastened, the jacket arms are crossed at the center front of the body and the strap from one sleeve extends around the back of the body, and can be fastened to the other sleeve.

Some known jackets have a crotch strap which extends downwardly from the front of the torso body or a jacket collar, under the patient's crotch, and upwardly to a fastening point on the jacket torso body rear. This crotch strap prohibits the wearer from pulling the jacket over his or her head and thus removing it.

It is an object of this invention to provide a straitjacket having a design which permits the jacket to be used with a variety of patients having a variety of clothing and body sizes and shapes.

It is another object of the invention to provide a straitjacket which permits the use of medical devices which require direct skin contact, such as EKG electrodes and ultrasound systems.

It is yet another object of the invention to provide a straitjacket which minimizes chafing and overheating of the patients, especially struggling patients.

Still another object of the invention is to provide a straitjacket the size and shape of which minimizes intimidation of the patient and other observers.

The further object of the intention is to provide a straitjacket which has a minimal cost to manufacture.

Other objects and advantages of the invention will be apparent upon reading the following detailed description and upon reference to the drawings. Throughout the drawings, like reference numerals refer to like parts.

### DESCRIPTION OF THE DRAWINGS

FIG. 1 is a front elevational view of the jacket as it appears when it is being worn by a patient.

FIG. 2 is a rear elevational view of the jacket as it appears when it is being worn by a patient.

FIG. 3 is a front elevational view of the jacket.

FIG. 4 is a rear elevational view of the jacket.

### DETAILED DESCRIPTION

While the invention will be described in connection with a preferred embodiment, it will be understood that it is not intended to limit the invention to this embodiment. On the contrary, it is intended to cover all alternatives, modifications and equivalents as may be included within the spirit and scope of the invention.

Turning to the drawings, there is shown a straitjacket 10 embodying the invention. The straitjacket can be made of any suitable material. In the illustrated embodiment of the present invention, a fabric having the characteristics of canvas is preferred.

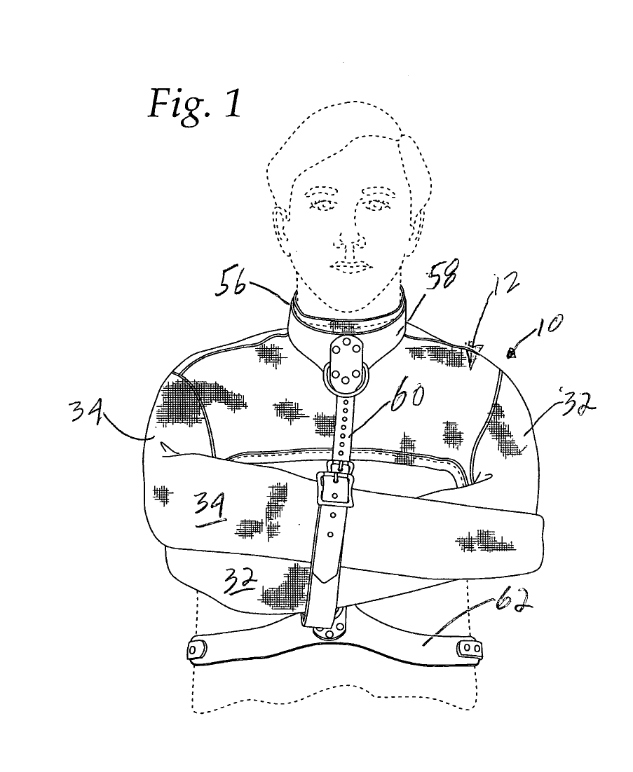

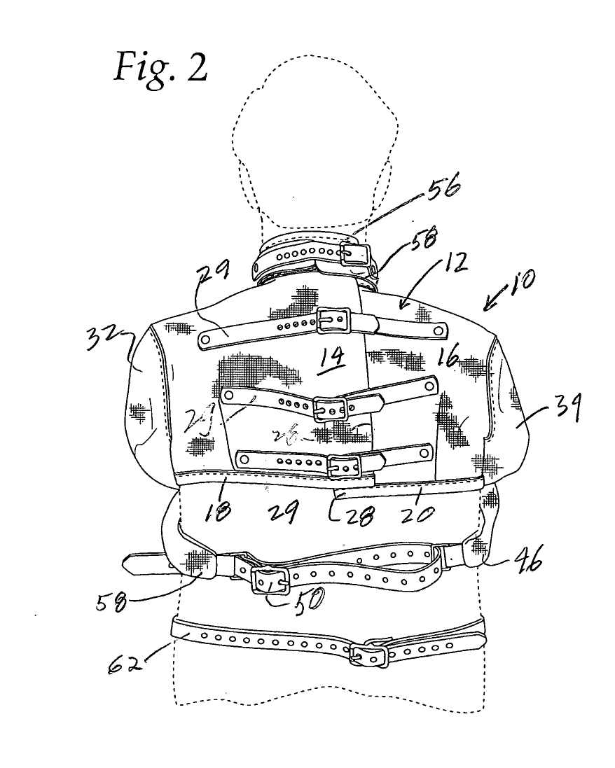

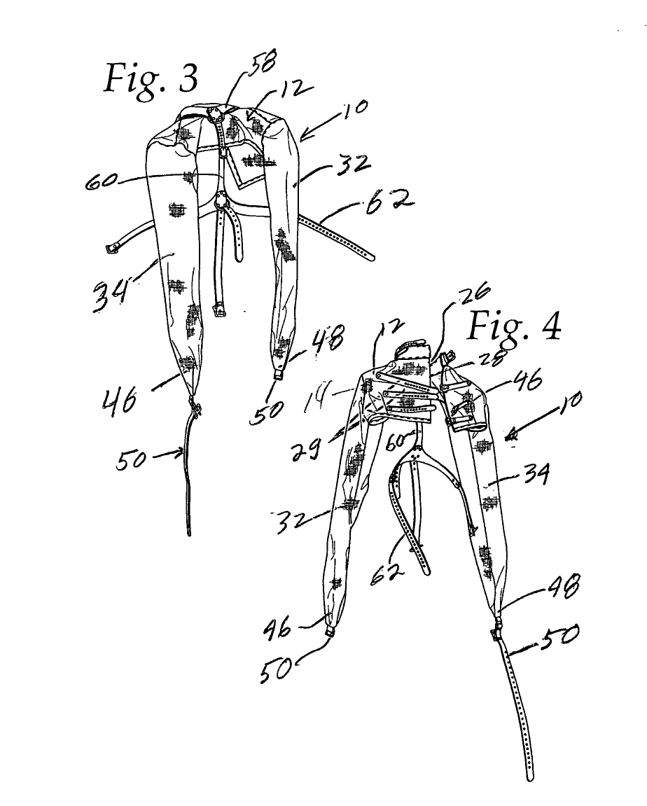

To enable the straitjacket 10 to fit a variety of patients having a variety of clothing sizes and body sizes and shapes in accordance with the invention, the straitjacket has a jacket torso body 12 which comprises at least two panels 14, 16. Each panel 14, 16 terminates at a lower edge 18, 20 adjacent to but below the wearer's under arms as shown in FIGS. 1 and 2. In a preferred embodiment, these lower edges 18, 20 are located about 2 inches below the wearers under arms, and, more specifically, the edges illustrated here are located 1⅜″ below the wearer's under arms. Thus, the straitjacket fabric covers only the wearer's upper back, upper chest, lower neck and arms. This permits the use of medical devices which require skin contact, such as EKG electrodes and ultrasound systems, in accordance with another aspect of the invention. In addition, the minimal aspect of the straitjacket minimizes chafing and overheating of patients, especially struggling patience; and may minimize the intimidation of a patient and other observers.

In accordance with a further aspect of the invention, the two panels 14, 16 each have respective back side edges 26 and 28. When the straitjacket is being worn, these edges 26 and 28 can be spaced apart from one another, or they can be configured so that the panels overlap so as to define a straitjacket partial body which will fit a wide variety of body shapes and sizes, in accordance with one aspect of the invention. One or more strap means 29 extend between the back panels 14, 16, and the straps 29 are provided with buckles or other known devices for adjusting the effective length of the straps 29 so as to snugly secure the straitjacket torso body around the patient.

Extending from the torso body 12 are sleeves 32, 34 which can be made of fabric similar to that comprising the torso body. Each sleeve has a length greater than the length of the wearer's arms, and the distal ends 46, 48 of each sleeve includes a fastening device 50 adapted to fasten the sleeve ends to one another and, if desired, to some other portion of the straitjacket so as to prohibit the straitjacket wearer from making uncontrolled movements of his or her arms. In the illustrated embodiment, this fastening device 50 comprises a belt and belt buckle, but other known fastening devices such as ropes can be used.

In accordance with yet another aspect of the invention, the sleeves can each have a length permitting the sleeves to be crossed at the center front of the wearer's body as suggested in FIG. 1, and then extended around the back of the wearer's body, when a car fastened to the other sleeve at a rear or posterior position. Thus, depending on the length of the sleeves, this sleeve interconnection can be located at the back or at the front of the patient.

The jacket includes a high annular collar 56, around which, if desired, can be placed an outer collar 58. These collars 56 and 58 can be made as an integral piece, and can be made integral with the jacket torso body 12, if desired.

To provide additional security, a chest strap 60 extends downwardly from the collar 58 across the wearer's anterior torso and, if desired, under the wearer's crotch. In accordance with one aspect of the invention, the chest strap 60 can be attached to a belt strap 62 adapted to be secured about the wearer's torso and further adapted to be attached to the chest strap at a posterior position. The chest strap 60 can also be attached to the belt strap 62 at an anterior position for additional security and effectiveness.

The following is claimed as invention:

1. A straightjacket adapted to fit wearers of more than one clothing size, the straitjacket comprising, in combination
a jacket torso body comprising at least two panels, each panel terminating at a lower edge located adjacent to but below the wearer's under arms,
sleeves each extending from the torso body to a distal end, each sleeve having a length greater than the length of the wearer's arms, the distal end of each sleeve including a fastening device,
an annular jacket collar affixed to the jacket torso body,
a chest strap adapted to extend downwardly from the collar and under the wearer's crotch,
and a belt strap adapted to be secured about the wearer's mid rib cage and further adapted to be attached to the chest strap at a posterior position.

2. A straitjacket according to claim 1 wherein the torso body has at least two back panels, the back panels having edges to define a straitjacket torso body rear opening,
at least one strap means extending between the back panels,
and means for adjusting the effective length of the strap means.

3. A straitjacket according to claim 2 wherein said sleeves each have a length permitting the sleeves to be crossed at the center front of the wearer's body, around the back of the wearer's body, and fastened to the other sleeve at a posterior position.

4. A straitjacket according to claim 1 wherein said belt strap is adapted to be attached to said chest strap at an anterior position.

5. A straitjacket according to claim 1 wherein said belt strap is adapted to be attached to said chest strap at a posterior position.

6. A straitjacket according to claim 1 wherein said torso panels terminate at lower edge is located about 2 inches below the wearers under arms.

7. A straitjacket according to claim 1 wherein said torso panels terminate at lower edge is located substantially 1⅜ inches below the wearers underarms.

8. A straitjacket according to claim 1 wherein said straitjacket covers only the wearers upper back, upper chest, lower neck and arms.
